(19) United States
(12) Patent Application Publication
(10) Pub. No.: US 2014/0039062 A1
(43) Pub. Date: Feb. 6, 2014

(54) INJECTABLE DEVICE AND METHOD FOR SCULPTING, AUGMENTING OR CORRECTING FACIAL FEATURES SUCH AS THE CHIN

(71) Applicant: Allergan, Inc., Irvine, CA (US)

(72) Inventors: Dimitrios Stroumpoulis, Annecy (FR); Ahmet Tezel, Fort Worth, TX (US)

(73) Assignee: Allergan, Inc, Irvine, CA (US)

(21) Appl. No.: 14/045,539

(22) Filed: Oct. 3, 2013

Related U.S. Application Data
(63) Continuation-in-part of application No. 13/206,454, filed on Aug. 9, 2011.
Application No. 13/206,454 is a continuation-in-part of application No. 12/910,466, filed on Oct. 22, 2010.
Application No. 12/910,466 is a continuation-in-part of application No. 12/178,574, filed on Jul. 23, 2008, now Pat. No. 8,318,695.
(60) Provisional application No. 61/710,470, filed on Oct. 5, 2012.
(60) Provisional application No. 60/952,770, filed on Jul. 30, 2007.

(51) Int. Cl. A61K 8/73; A61K 31/167

(52) U.S. Cl. CPC A61K 8/735; A61K 31/167
USPC 514/626; 536/53; 514/777

(57) ABSTRACT

An injectable device useful for facial sculpting and correction of facial features, for example, for augmenting the chin in a human being is provided, the device being made of a composition comprising a hyaluronic acid crosslinked with a multifunctional polyethylene glycol (PEG)-based crosslinking agent.

This application claims the benefit of, and priority to, U.S. Provisional Patent Application No. 61/710,470, filed Oct. 5, 2012, and is also a continuation-in-part of U.S. patent application Ser. No. 13/206,454, filed Aug. 9, 2011, which is a continuation-in-part of U.S. patent application Ser. No. 12/910,466, filed Oct. 22, 2010, which is a continuation-in-part of U.S. patent application Ser. No. 12/178,574, filed Jul. 23, 2008, now U.S. Pat. No. 8,318,695, which claims priority to U.S. Provisional Application No. 60/952,770, filed Jul. 30, 2007, the entire disclosure of each of these applications being incorporated herein by this reference.

The present invention generally relates to implantable prosthesis and more specifically relates to an injectable device for adding structure and contour to the lower face.

In June 2006, 3 members of the Juvéderm family of hyaluronic acid (HA)-based dermal fillers, Juvéderm 30, Juvéderm Ultra, and Juvéderm Ultra Plus Injectable Gels (Allergan Inc., Irvine, Calif.), were approved by the U.S. Food and Drug Administration (FDA) for the correction of moderate to severe facial wrinkles and folds such as nasolabial folds. In January 2010, FDA approved the addition of 0.3% w/w of lidocaine into the Juvéderm formulations (Juvéderm Ultra XC and Juvéderm Ultra Plus XC) to decrease pain associated with the procedure. All members of the Juvéderm family are currently made from essentially the same raw materials: hyaluronic acid crosslinked with 1, 4-butanediol diglycidyl ether (BDDE) in physiological buffer.

Dermal fillers are sometimes used by cosmetic and reconstructive surgeons as a way to add more volume to the face, rather than to simply correct wrinkles and folds. Hyaluronic acid is still considered the most desirable dermal filler in that it does not pose the risk of an allergic reaction and it is temporary. The great majority of hyaluronic acid-based dermal fillers were developed specifically for treating wrinkles and folds in skin, not for facial contouring. Current HA-based fillers are limited for this indication because the dermal filler formulations lack adequate ability to lift while providing long-lasting results in this anatomic area. There is a great need for an injectable hyaluronic acid based dermal filler that is specifically designed to be effective in adding substantial volume to the face, for example, for contouring the lower face, for example, for augmenting or correcting the chin, for example, for correction of chin retrusion.

The shape of the chin has long been recognized as an important feature of the face that elicits a strong aesthetic perception that tends to be associated with personality traits of an individual. A deficient chin that lacks projection is commonly labeled a “weak chin” while prominent chins are labeled “strong chins”, both implying strength of personality.

Several studies have suggested that faces with average proportions are viewed as the most attractive and that small features including a small chin are interpreted as attractive in females while the expanded chin and jaw, as a result of maturation, are interpreted as attractive in males. The appearance of the chin is a determinant of perceived attractiveness and can even influence an individual's psychosocial well-being.

Chin augmentation is generally performed not through the use of dermal fillers designed for treating wrinkles and fold lines in the skin, but by surgically placing a permanent implant above the jaw. The procedure is currently among the top aesthetic surgical procedures performed, based on the American Society for Aesthetic Plastic Surgery (ASAPS), and has increased 71% from 2010.

### SUMMARY OF THE INVENTION

Accordingly, an injectable device is provided for facial sculpturing, for example, for augmenting or correcting the chin in a human being.

The device generally comprises a composition comprising a hyaluronic acid (HA), preferably a low molecular weight HA, crosslinked with a non-BDDE, multifunctional crosslinking agent, for example, a multifunctional polyethylene glycol (PEG)-based crosslinking agent, the composition being suitable for injection and capable of augmenting and providing substantial volume, lift and/or sculpturing of the lower face, for example, the chin.

The multifunctional PEG-based crosslinking agent may be a bifunctional PEG-based crosslinking agent, a trifunctional PEG-based crosslinking agent, a tetrafunctional PEG-based crosslinking agent, a pentafunctional PEG-based crosslinking agent, a hexafunctional PEG-based crosslinking agent, a heptafunctional PEG-based crosslinking agent, an octafunctional PEG-based crosslinking agent, a nonafunctional PEG-based crosslinking agent, or a decafunctional PEG-based crosslinking agent.

In some embodiments, the crosslinking agent is a tetrafunctional polyethylene glycol (PEG)-crosslinking agent. In one embodiment, the tetrafunctional crosslinker is pentaerythritol tetraglycidyl ester.

In one aspect of the invention, the hyaluronic acid, prior to crosslinking, is substantially entirely, or 100%, low molecular weight hyaluronic acid, for example, a HA having a mean molecular weight of between about 300,000 Da and about 840,000 Da.

It has been surprisingly found that using a low molecular weight HA crosslinked with the multifunctional polyethylene glycol based crosslinking agent, rather than a high molecular weight HA or a mixture of high and low molecular weight HA crosslinked with BDDE has resulted in a more robust, longer lasting hydrogel, having a higher viscosity and elasticity, and more specifically suitable for facial sculpturing and augmentation by means of subcutaneous or supraperiosteal injection.

In one embodiment, the composition has an HA concentration of between about 20 mg/g and about 30 mg/g, for example, an HA concentration of about 22 mg/g, for example, about 23 mg/g, for example, about 24 mg/g, for example, about 25 mg/g, for example, about 26 mg/g, for example, about 27 mg/g, for example, about 28 mg/g, for example, about 29 mg/g, for example, about 30 mg/g. In a more specific embodiment, the composition has an HA concentration of between 22.5 mg/g to 27.5 mg/g, for example, 25.0 mg/g, or for example 23.5 mg/g.

In another aspect of the invention, the composition comprises a multifunctional PEG-crosslinked hyaluronic acid, the composition including between about 5% and about 25% crosslinking agent/NaHA w/w, for example, between about 10% and about 20% of the crosslinking agent, for example, about 12%, about 13%, or about 15% crosslinking agent/NaHA, and the crosslinking agent is, for example, pentaerythritol tetra glycidyl ether.

In a specific embodiment, the compositions comprises low molecular weight hyaluronic acid (NaHA) crosslinked with about 13% of pentaerythritol tetra glycidyl ether (PEGE)/NaHA (w/w), and formulated to a concentration of about 25 mg/g with 0.3% lidocaine hydrochloride (w/w) in a phosphate buffer.

In another aspect of the invention, methods are provide for contouring or correcting a facial feature of an individual, the methods comprising the step of subdermally administering into the facial feature of the patient, an effective amount, for example, about 1.0 ml, or more, for example, about 2.0 ml or more, for example, about 3.0 ml or more, of a composition of the invention. The facial feature may be a chin, for example, a retruded chin of a patient.

In another aspect of the invention, methods are provided for correcting a retruded chin of a patient, the method comprising supraperiostally administering in the chin of the patient, an effective amount of a composition comprising a low molecular weight hyaluronic acid crosslinked with a PEG-based, non-BDDE crosslinker, for example, a pentaerythritol tetraglycidyl ester crosslinker.

Each and every feature described herein, and each and every combination of two or more of such features, is included within the scope of the present invention provided that the features included in such a combination are not mutually inconsistent.

### DETAILED DESCRIPTION

Aspects of the present specification disclose, in part, a polyethylene glycol (PEG)-based crosslinking agent. As used herein, the term “PEG-based crosslinking agent” is synonymous with “PEG-based crosslinker” and refers to a PEG molecule comprising at least two reactive sites useful to covalently conjugate another molecule to the PEG molecule. PEG comprises a group of biocompatible, hydrophilic, and inert polymers having the general formula HO(CH₂CH₂O)ₙH, where n is an integer from 2 to 100, which is synthesized by the polymerization of ethylene oxide. A PEG molecule can be linear or branched. Branched PEGs include, without limitation, forked PEGs, star PEGs, comb PEGs, brush PEGs, and graft PEGs. A forked PEG is a branched PEG comprising two polymer chains emanating from a single branch point. A star PEG is a branched PEG comprising three or more linear polymer chains emanating from a central core group or a single branch point. A comb PEG is a branched PEG comprising two or more three-way branch points and linear side chains emanating from a main backbone polymer chain. A brush PEG is a branched PEG comprising three or more linear polymer chains emanating from a main backbone polymer chain. A graft PEG is a branched PEG comprising two or more polymer chains where one or more polymer chains are different, structurally or configurationally, from the main chain. The polymer chains comprising a PEG may be blocked.

In standard nomenclature, a branched PEG can be referred to by the number of polymer chains is comprises. Thus, a branched PEG having three polymer chains is referred to as a three-arm PEG or 3-arm PEG, a branched PEG having four polymer chains is referred to as a four-arm PEG or 4-arm PEG, a branched PEG having five polymer chains is referred to as a five-arm PEG or 5-arm PEG, a branched PEG having six polymer chains is referred to as a six-arm PEG or 6-arm PEG, a branched PEG having seven polymer chains is referred to as a seven-arm PEG or 7-arm PEG, etc. The physical properties of PEG, such as melting point, cohesiveness, and viscosity, can be altered by varying the length of the polymer chain, the type of initiator used during the polymerization process, and/or whether the PEG has a linear or branched configuration. PEG molecules, both linear and branched, are commercially available over a wide range of molecular weights from 300 g/mol to 10,000,000 g/mol.

A polymer chain of a PEG-based crosslinking agent may be functional in that it comprises a reactive site used to conjugate the PEG chain to another molecule. A PEG-based crosslinker containing more than one reactive site is referred to generally as a multifunctional PEG-based crosslinker, or more specifically by the number of reactive sites it contains. For example, a bifunctional PEG-based crosslinker has two reactive sites useful for crosslinking purposes, a trifunctional PEG-based crosslinker has three reactive sites useful for crosslinking purposes, a tetrafunctional PEG-based crosslinker has four reactive sites useful for crosslinking purposes, a pentafunctional PEG-based crosslinker has five reactive sites useful for crosslinking purposes, a hexafunctional PEG-based crosslinker has six reactive sites useful for crosslinking purposes, a heptafunctional PEG-based crosslinker has seven reactive sites useful for crosslinking purposes, etc. The number of functional sites on a PEG-based crosslinker disclosed herein is limited only by the ability of the hyaluronic acid polymer strands to bind to the resulting active sites on the crosslinker due to, e.g., geometry and steric hindrance.

A polymer chain of a PEG-based crosslinking agent is made functional by attaching a reactive group to the free end of a polymer chain from a base PEG molecule. Any reactive group that can be used to covalently join glycosaminoglycan polymers to the PEG-based crosslinker may be used, including, without limitation, epoxides. The PEG-based crosslinking agents disclosed herein may be made according to any PEG synthesis methods known to one of ordinary skill in the art. Generally, a multifunctional PEG-based crosslinking agent is synthesized from a base poly-alcohol or PEG molecule having the desired chain length and branching by attaching epoxide groups. Such epoxide groups can be attached to the base poly-alcohol or PEG molecule by deprotonating the hydroxyl groups and reacting with epichlorohydrin. Example 1 describes the synthesis of a specific PEG-based crosslinking agent disclosed herein.

A PEG-based crosslinker may have a variety of polymer chain lengths which affect its mechanical properties. As such, a PEG-based crosslinking agent disclosed herein is of tunable size. In an aspect of this embodiment, a trifunctional PEG-based crosslinking agent comprises three polymer chains emanating from a central core group, with each chain having the structure CH₂(OCH₂CH₂)ₙOCH₂ epoxide, where n is an integer from 0 to 60. In another aspect of this embodiment, a tetrafunctional PEG-based crosslinking agent comprises four polymer chains emanating from a central core group with each chain having the structure CH₂(OCH₂CH₂)ₙOCH₂ epoxide, where n is an integer from 0 to 60. In yet another aspect of this embodiment, a pentafunctional PEG-based crosslinking agent comprises five polymer chains emanating from a central core group, with each chain having the structure CH₂(OCH₂CH₂)ₙOCH₂ epoxide, where n is an integer from 0 to 60. In still another aspect of this embodiment, a hexafunctional PEG-based crosslinking agent comprises six polymer chains emanating from a central core group, with each chain having the structure CH₂(OCH₂CH₂)ₙOCH₂ epoxide, where n is an integer from 0 to 60. In a further aspect of this embodiment, a heptafunctional PEG-based crosslinking agent comprises seven polymer chains emanating from a central core group, with each chain having the structure CH₂(OCH₂CH₂)ₙOCH₂ epoxide, where n is an integer from 0 to 60. In a yet further aspect of this embodiment, an octafunctional PEG-based crosslinking agent comprises eight polymer chains emanating from a central core group, with each chain having the structure CH₂(OCH₂CH₂)ₙOCH₂ epoxide, where n is an integer from 0 to 60. In a still further aspect of this embodiment, a nonafunctional PEG-based crosslinking agent comprises nine polymer chains emanating from a central core group, with each chain having the structure CH₂(OCH₂CH₂)ₙOCH₂ epoxide, where n is an integer from 0 to 60. In another aspect of this embodiment, a decafunctional PEG-based crosslinking agent comprises ten polymer chains emanating from a central core group, with each chain having the structure CH₂(OCH₂CH₂)ₙOCH₂ epoxide, where n is an integer from 0 to 60. A core group can be a carbon atom, a generational carbon like a first or second generation carbon, or a dendrite.

In another aspect of this embodiment, a trifunctional PEG-based crosslinking agent comprises three polymer chains emanating from a PEG polymer backbone having the structure HO(CH₂CH₂O)ₘH, where m is an integer from 2 to 100, with each chain having the structure CH₂(OCH₂CH₂)ₙOCH₂ epoxide, where n is an integer from 0 to 60. In another aspect of this embodiment, a tetrafunctional PEG-based crosslinking agent comprises four polymer chains emanating from a PEG polymer backbone having the structure HO(CH₂CH₂O)ₘH, where m is an integer from 2 to 100, with each chain having the structure CH₂(OCH₂CH₂)ₙOCH₂ epoxide, where n is an integer from 0 to 60. In yet another aspect of this embodiment, a pentafunctional PEG-based crosslinking agent comprises five polymer chains emanating from a PEG polymer backbone having the structure HO(CH₂CH₂O)ₘH, where m is an integer from 2 to 100, with each chain having the structure CH₂(OCH₂CH₂)ₙOCH₂ epoxide, where n is an integer from 0 to 60. In still another aspect of this embodiment, a hexafunctional PEG-based crosslinking agent comprises six polymer chains emanating from a PEG polymer backbone having the structure HO(CH₂CH₂O)ₘH, where m is an integer from 2 to 100, with each chain having the structure CH₂(OCH₂CH₂)ₙOCH₂ epoxide, where n is an integer from 0 to 60. In a further aspect of this embodiment, a heptafunctional PEG-based crosslinking agent comprises seven polymer chains emanating from a PEG polymer backbone having the structure HO(CH₂CH₂O)ₘH, where m is an integer from 0 to 100, with each chain having the structure CH₂(OCH₂CH₂)ₙOCH₂ epoxide, where n is an integer from 0 to 60. In a yet further aspect of this embodiment, an octafunctional PEG-based crosslinking agent comprises eight polymer chains emanating from a PEG polymer backbone having the structure HO(CH₂CH₂O)ₘH, where m is an integer from 2 to 100, with each chain having the structure CH₂(OCH₂CH₂)ₙOCH₂ epoxide, where n is an integer from 0 to 60. In a still further aspect of this embodiment, a nonafunctional PEG-based crosslinking agent comprises nine polymer chains emanating from a PEG polymer backbone having the structure HO(CH₂CH₂O)ₘH, where m is an integer from 2 to 100, with each chain having the structure CH₂(OCH₂CH₂)ₙOCH₂ epoxide, where n is an integer from 0 to 60. In another aspect of this embodiment, a decafunctional PEG-based crosslinking agent comprises ten polymer chains emanating from a PEG polymer backbone having the structure HO(CH₂CH₂O)ₘH, where m is an integer from 2 to 100, with each chain having the structure CH₂(OCH₂CH₂)ₙOCH₂ epoxide, where n is an integer from 0 to 60.

In another aspect of this embodiment, a bifunctional PEG-based crosslinking agent has a molecular weight of between about 200 Da to about 10,000 Da. In yet another aspect of this embodiment, a trifunctional PEG-based crosslinking agent has a molecular weight of between about 200 Da to about 10,000 Da. In still another aspect of this embodiment, a tetrafunctional PEG-based crosslinking agent has a molecular weight of between about 200 Da to about 10,000 Da. In a further aspect of this embodiment, a pentafunctional PEG-based crosslinking agent has a molecular weight of between about 200 Da to about 10,000 Da. In another aspect of this embodiment, a hexafunctional PEG-based crosslinking agent has a molecular weight of between about 200 Da to about 10,000 Da.

Matrix polymers, such as e.g., polysaccharides polymers like glycosaminoglycan polymers, may be crosslinked with only one type of multifunctional PEG-based crosslinker or with two or more different types of multifunctional PEG-based crosslinkers. In an aspect of this embodiment, glycosaminoglycan polymer strands may be crosslinked solely with a trifunctional PEG-based crosslinker, a tetrafunctional PEG-based crosslinker, a pentafunctional PEG-based crosslinker, a hexafunctional PEG-based crosslinker, a heptafunctional PEG-based crosslinker, an octafunctional PEG-based crosslinker, a nonafunctional PEG-based crosslinker, or a decafunctional PEG-based crosslinker. In other aspects of this embodiment, glycosaminoglycan polymer strands may be crosslinked using a combination of, e.g., trifunctional and tetrafunctional PEG-based crosslinkers, trifunctional and pentafunctional PEG-based crosslinkers, tetrafunctional and pentafunctional PEG-based crosslinkers, tetrafunctional and hexafunctional PEG-based crosslinkers, tetrafunctional and octafunctional PEG-based crosslinkers, pentafunctional and hexafunctional PEG-based crosslinkers, pentafunctional and hepafunctional PEG-based crosslinkers, or pentafunctional and nonafunctional PEG-based crosslinkers. By selecting the multifuntionality of the PEG-based crosslinkers and/or varying the amounts of the different types of multifunctional PEG-based crosslinkers, the mechanical strength of the resulting hydrogel can be tailored to the desired specifications (see, e.g., Examples 5, 6, and 7).

Matrix polymers, such as e.g., polysaccharides polymers like glycosaminoglycan polymers, may be crosslinked solely with the multifunctional PEG-based crosslinkers disclosed herein or in combination with any other crosslinking agent suitable for making crosslinked hyaluronan. Non limiting examples of such crosslinking agents include dialdehydes and disufides crosslinking agents including, without limitation, divinyl sulfones, diglycidyl ethers, and bis-epoxides. Non-limiting examples of hyaluronan crosslinking agents include divinyl sulfone (DVS), 1,4-butanediol diglycidyl ether (BDDE), 1,2-bis(2,3-epoxypropoxy)ethylene (EGDGE), 1,2,7,8-diepoxyoctane (DEO), biscarbodiimide (BCDI), adipic dihydrazide (ADH), bis(sulfosuccinimidyl)suberate (BS), hexamethylenediamine (HMDA), 1-(2,3-epoxypropyl)-2,3-epoxycyclohexane, or combinations thereof. By mixing a PEG-based crosslinker disclosed herein with another crosslinker, such as, e.g., the ones disclosed herein, in varying ratios, the mechanical strength and hardness of the final hyaluronan composition may be tuned as desired (see, e.g., Examples 5, 6, and 7).

In one aspect of this embodiment, glycosaminoglycan polymers are crosslinked using a combination of PEG-based crosslinkers disclosed herein and BDDE. In another aspect of this embodiment, glycosaminoglycan polymer strands are crosslinked using a combination of PEG-based crosslinkers disclosed herein and EGDGE. In yet another aspect of this embodiment, glycosaminoglycan polymer strands are crosslinked using a combination of PEG-based crosslinkers disclosed herein and DEO. In still another aspect of this embodiment, glycosaminoglycan polymer strands are crosslinked using a combination of PEG-based crosslinkers disclosed herein and DVS.

Matrix polymers, such as e.g., polysaccharides polymers like glycosaminoglycan polymers, are crosslinked using the PEG-based crosslinking agents disclosed herein using conventional procedures known to a person of ordinary skill. For example, glycosaminoglycan polymers are brought into contact with a PEG-based crosslinker and allowed to react. The glycosaminoglycan polymers may be reacted with more than one PEG-based crosslinker as disclosed herein in either a step-wise fashion, with a lower functionality PEG-based crosslinker being brought into contact first or with a higher functionality PEG-based crosslinker being brought into contact first. Alternatively, glycosaminoglycan polymers may be reacted with a plurality of PEG-based crosslinkers in one step.

Matrix polymers, such as e.g., polysaccharides polymers like glycosaminoglycan polymers, that may be crosslinked using the PEG-based crosslinking agents and methods disclosed herein. Additional matrix polymers, such as e.g., polysaccharides polymers like glycosaminoglycan polymers, that may be crosslinked using the PEG-based crosslinking agents and methods disclosed herein are described in, e.g., Piron and Tholin, Polysaccharide Crosslinking, Hydrogel Preparation, Resulting Polysaccharides(s) and Hydrogel(s), uses Thereof, U.S. Patent Publication 2003/0148995; Lebreton, Cross-Linking of Low and High Molecular Weight Polysaccharides Preparation of Injectable Monophase Hydrogels and Polysaccharides and Hydrogels thus Obtained, U.S. Patent Publication 2006/0194758; Lebreton, Viscoelastic Solutions Containing Sodium Hyaluronate and Hydroxypropyl Methyl Cellulose, Preparation and Uses, U.S. Patent Publication 2008/0089918; Stroumpoulis, et al., Polysaccharide Gel Formulations Having Increased Longevity, U.S. Patent Publication 2009/014333; Stroumpoulis, et al., Polysaccharide Gel Formulations Having Increased Longevity, U.S. Patent Publication 2010/0004198; Lebreton, Hyaluronic Acid-Based Gels Including Lidocaine, U.S. Patent Publication 2010/0028438; Stroumpoulis, et al., Polysaccharide Gel Formulations Having Multistage Bioactive Agent Delivery, U.S. Patent Publication 2010/0098764; and Di Napoli, Composition and Method for Intradermal Soft Tissue Augmentation, International Patent Publication WO 2004/073759, each of which is hereby incorporated by reference in its entirety.

Any conventional crosslinking method may be used to crosslink glycosaminoglycan polymers using a multifunctional PEG-based crosslinker disclosed herein alone, with another type of multifunctional PEG-based crosslinker, and/or with conjunction with a non-PEG-based crosslinker. Generally, a matrix polymer undergoes a preparation step and then is simply mixed with a crosslinker in order to initiate the crosslinking reaction. For example, a glycosaminoglycan is first hydrated by mixing the polymer with a 0.01-1% sodium hydroxide solution and incubating at ambient temperature for about 1 hour to about 5 hours. Next, about 10% (w/w) to about 25% (w/w), or about 50 mg to about 2,000 mg, of an appropriate multifunctional PEG-based crosslinking agent(s) (about 200 Da to about 10,000 Da) is added to the hydrated glycosaminoglycan. If a non-PEG-based crosslinker is also employed, about 20 to about 200 mg of non-PEG-based crosslinker is added as well. The mixture is then mechanically homogenized, and then placed in an about 40 to about 70° C. oven for about 1 hour to about 10 hours. The resulting crosslinked hydrogel is neutralized with an equimolar amount of hydrochloric acid and swelled in a physiologically-acceptable solution, such as, e.g., a buffered solution of about pH 5.5 to about pH 8.5.

In one aspect of this embodiment, a crosslinking reaction comprises about 90% (w/w) glycosaminoglycan polymer and about 10% multifunctional PEG-based crosslinking agent, a crosslinking reaction comprises about 89% (w/w) glycosaminoglycan polymer and about 11% multifunctional PEG-based crosslinking agent, a crosslinking reaction comprises about 88% (w/w) glycosaminoglycan polymer and about 12% multifunctional PEG-based crosslinking agent, a crosslinking reaction comprises about 87% (w/w) glycosaminoglycan polymer and about 13% multifunctional PEG-based crosslinking agent, a crosslinking reaction comprises about 86% (w/w) glycosaminoglycan polymer and about 14% multifunctional PEG-based crosslinking agent, a crosslinking reaction comprises about 85% (w/w) glycosaminoglycan polymer and about 15% multifunctional PEG-based crosslinking agent, a crosslinking reaction comprises about 84% (w/w) glycosaminoglycan polymer and about 16% multifunctional PEG-based crosslinking agent, a crosslinking reaction comprises about 83% (w/w) glycosaminoglycan polymer and about 17% multifunctional PEG-based crosslinking agent, a crosslinking reaction comprises about 82% (w/w) glycosaminoglycan polymer and about 18% multifunctional PEG-based crosslinking agent, a crosslinking reaction comprises about 81% (w/w) glycosaminoglycan polymer and about 19% multifunctional PEG-based crosslinking agent, or a crosslinking reaction comprises about 80% (w/w) glycosaminoglycan polymer and about 20% multifunctional PEG-based crosslinking agent.

In another aspect of this embodiment, a crosslinking reaction comprises about 90% (w/w) glycosaminoglycan polymer and about 10% pentaerythritol tetraglycidyl ether (PEGE) crosslinking agent, a crosslinking reaction comprises about 89% (w/w) glycosaminoglycan polymer and about 11% PEGE crosslinking agent, a crosslinking reaction comprises about 88% (w/w) glycosaminoglycan polymer and about 12% PEGE, a crosslinking reaction comprises about 87% (w/w) glycosaminoglycan polymer and about 13% PEGE, a crosslinking reaction comprises about 86% (w/w) glycosaminoglycan polymer and about 14% PEGE, a crosslinking reaction comprises about 85% (w/w) glycosaminoglycan polymer and about 15% PEGE, a crosslinking reaction comprises about 84% (w/w) glycosaminoglycan polymer and about 16% PEGE, a crosslinking reaction comprises about 83% (w/w) glycosaminoglycan polymer and about 17% multifunctional PEGE, a crosslinking reaction comprises about 82% (w/w) glycosaminoglycan polymer and about 18% PEGE, a crosslinking reaction comprises about 81% (w/w) glycosaminoglycan polymer and about 19% PEGE, or a crosslinking reaction comprises about 80% (w/w) glycosaminoglycan polymer and about 20% PEGE.

In some especially advantageous embodiments, the composition is crosslinked with between about 10% and about 20% pentaerythritol tetra glycidyl ether (PEGE)/NaHA (w/w), for example, about 13% pentaerythritol tetra glycidyl ether (PEGE)/NaHA (w/w).

In a specific embodiment of the invention, the hyaluronan polymer has a mean molecular weight of between about 310,000 Da and about 840,000 Da and is crosslinked with PEGE. The initial PEGE crosslinking reaction is about 10% to about 15% (w/w), for example, about 13% PEGE (w/w). The concentration of NaHA in the final composition is about 20 to about 30 mg HA/g (mg/g).

Aspects of the present specification provide, in part, a hydrogel composition comprising glycosaminoglycan polymers. As used herein, the term “glycosaminoglycan” is synonymous with “GAG” and “mucopolysaccharide” and refers to long unbranched polysaccharides consisting of a repeating disaccharide units. The repeating unit consists of a hexose (six-carbon sugar) or a hexuronic acid, linked to a hexosamine (six-carbon sugar containing nitrogen) and pharmaceutically acceptable salts thereof. Members of the GAG family vary in the type of hexosamine, hexose or hexuronic acid unit they contain, such as, e.g., glucuronic acid, iduronic acid, galactose, galactosamine, glucosamine) and may also vary in the geometry of the glycosidic linkage. Any glycosaminoglycan is useful in the compositions disclosed herein with the proviso that the glycosaminoglycan improves a soft tissue condition as disclosed herein. GAGs useful in the compositions and methods disclosed herein are commercially available.

Aspects of the present specification provide, in part, a hydrogel composition comprising a hyaluronan. As used herein, the term “hyaluronan” is synonymous with “hyaluronic acid”, “HA”, “hyaluronic acid” and “hyaluronate”. Hyaluronan includes anionic, non-sulfated glycosaminoglycan polymers comprising disaccharide units, which themselves include D-glucuronic acid and D-N-acetylglucosamine monomers, linked together via alternating β-1,4 and β-1,3 glycosidic bonds and pharmaceutically acceptable salts thereof. Hyaluronan can be purified from animal and non-animal sources. Polymers of hyaluronan can range in size from about 5,000 Da to about 20,000,000 Da. Any hyaluronan is useful in the compositions disclosed herein with the proviso that the hyaluronan improves a soft tissue condition as disclosed herein. Non-limiting examples of pharmaceutically acceptable salts of hyaluronan include sodium hyaluronate, potassium hyaluronate, magnesium hyaluronate, calcium hyaluronate, and combinations thereof.

Aspects of the present specification provide, in part, a hydrogel composition comprising crosslinked glycosaminoglycan polymers having a degree of crosslinking. As used herein, the term “degree of crosslinking” refers to the percentage of monomeric units of a glycosaminoglycan polymer that are bound to a cross-linking agent, such as, e.g., the disaccharide monomer units of hyaluronan. Thus, a hydrogel composition comprising crosslinked glycosaminoglycan polymers with a 4% degree of crosslinking means that on average there are four crosslinking molecules for every 100 monomeric units. Every other parameter being equal, the greater the degree of crosslinking, the harder a composition comprising crosslinked glycosaminoglycan polymers becomes.

In other aspects of this embodiment, a hydrogel composition comprises crosslinked glycosaminoglycan polymers where the degree of crosslinking is between about 1% and about 40%, for example, between about 2% and about 38%, for example, between about 4% and about 36%, for example, between about 6% and about 34%, for example, between about 8% and about 32% for example, between about 10% and about 30% for example, between about 12% and about 28%, for example, between about 14% and about 26% for example, between about 16% and about 24% for example, between about 18% and about 22%, for example, about 20%.

In other aspects of this invention, at least a portion of the HA in the compositions may be uncrosslinked. As used herein, the term “uncrosslinked” refers to a lack of intermolecular bonds joining the individual matrix polymer molecules, or monomer chains. As such, an uncrosslinked glycosaminoglycan polymer is not linked to any other glycosaminoglycan polymers by an intermolecular bond. For example, the compositions may comprise uncrosslinked glycosaminoglycan polymers where the uncrosslinked glycosaminoglycan polymers represents, e.g., about 20% or less by weight, about 18% or less by weight, about 15% or less by weight, about 12% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, of the total amount of glycosaminoglycan polymers present in the composition. In yet other aspects of this embodiment, a hydrogel composition comprises uncrosslinked glycosaminoglycan polymers where the uncrosslinked glycosaminoglycan polymers represents, e.g., about 10% to about 20% by weight, about 10% to about 15% by weight, about 5% to about 20% by weight, about 5% to about 15% by weight, about 5% to about 10% by weight, about 2% to about 20% by weight, about 2% to about 15% by weight, about 2% to about 10% by weight, or about 2% to about 5% by weight, of the total amount of glycosaminoglycan polymers present in the composition.

In another aspect, the compositions are made of substantially entirely low molecular weight hyaluronan polymers prior to the crosslinking, that is at least about 90% or more, by weight, of the hyaluronic acid in the compositions is what is referred to as low molecular weight hyaluronic acid. As used herein, the term “low molecular weight hyaluronan polymer” or “low molecular weight hyaluronan” refers to a hyaluronan polymer that has a molecular weight of less than 1,000,000 Da, more specifically, about 900,000 Da or less. Such low molecular weight hyaluronan polymers include a hyaluronan polymers of about 200,000 Da, about 300,000 Da, about 400,000 Da, about 500,000 Da, about 600,000 Da, about 700,000 Da, about 800,000 Da, or about 900,000 Da. In some embodiments of the invention, all of the hyaluronic acid in the compositions, that is 100% of the hyaluronic acid in the compositions, comprises such low molecular weight hyaluronic acid having a mean molecular weight of between about 300,000 Da and about 900,000 Da.

In some embodiments, the compositions comprise at least some high molecular weight hyaluronan polymers. As used herein, the term “high molecular weight hyaluronan polymer” or “high molecular weight hyaluronan” refers to a hyaluronan polymer that has a molecular weight of 1,000,000 Da or greater. Non-limiting examples of a high molecular weight hyaluronan polymer include a hyaluronan polymer of about 1,500,000 Da, about 2,000,000 Da, about 2,500,000 Da, about 3,000,000 Da, about 3,500,000 Da, about 4,000,000 Da, about 4,500,000 Da, or about 5,000,000 Da.

In aspects of this embodiment, the compositions are made by the process of crosslinking the low molecular weight hyaluronic acid with a tetrafunctional PEG crosslinking agent to form a highly viscous gel, then sizing the gel by passing the material through a screen (e.g. mesh size of 25 μm, a 43 μm, a 60 μm, a 100 μm, or 105 μm mesh size) only one time prior to sterilization and packaging in syringes for use. In a specific embodiment, the bulk gel material is sized by passing the material no more than a single time, through a mesh having a pore size of about 100 μm, prior to sterilization and packaging.

Alternatively, the gel may be sized in a more conventional manner used for sizing conventional HA-BDDE based dermal fillers by passing the gel through a mesh a plurality of times. In aspects of this embodiment, the composition is sized by passing the material through a 25 μm, a 43 μm, a 60 μm, or a 105 μm, mesh screen twice, three times, four times, five times, six times, seven times, eight times, nine times, or ten times.

Aspects of the present specification provide, in part, a hydrogel composition that may, or may not, comprise an anesthetic agent. An anesthetic agent is preferably a local anesthetic agent, i.e., an anesthetic agent that causes a reversible local anesthesia and a loss of nociception, such as, e.g., aminoamide local anesthetics and aminoester local anesthetics. The amount of an anesthetic agent included in a hydrogel composition disclosed herein is an amount effective to mitigate pain experienced by an individual upon administration of the composition. As such, the amount of an anesthetic agent included in a hydrogel composition disclosed herein is between about 0.1% (w/w) to about 5% (w/w) by weight of the total composition. Non-limiting examples of anesthetic agents include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, combinations thereof, and salts thereof. A non-limiting example of a combination local anesthetic is lidocaine/prilocaine (EMLA).

In a specific embodiment of the invention, the composition includes a lidocaine, for example, lidocaine chlorhydrate at an effective concentration so as to reduce pain upon injection. In this embodiment, the composition may include lidocaine at a concentration of between about 0.27% to about 0.33% w/w, or more specifically about 0.30% w/w.

Aspects of the present specification provide, in part, a hydrogel composition comprising crosslinked glycosaminoglycan polymers as disclosed herein that exhibits a complex modulus, an elastic modulus, a viscous modulus and/or a tan δ. The compositions as disclosed herein are viscoelastic in that the composition has an elastic component (solid-like such as, e.g., crosslinked glycosaminoglycan polymers) and a viscous component (liquid-like such as, e.g., uncrosslinked glycosaminoglycan polymers or a carrier phase) when a force is applied (stress, deformation). The rheological attribute that described this property is the complex modulus (G*), which defines a composition's total resistance to deformation. The complex modulus can be defined as the sum of the elastic modulus (G′) and the viscous modulus (G″). Falcone, et al., Temporary Polysaccharide Dermal Fillers: A Model for Persistence Based on Physical Properties, Dermatol Surg. 35(8): 1238-1243 (2009); Tezel, supra, 2008; Kablik, supra, 2009; Beasley, supra, 2009; each of which is hereby incorporated by reference in its entirety. Elastic modulus characterizes the firmness of a composition and is also known as the storage modulus because it describes the storage of energy from the motion of the composition. The elastic modulus describes the interaction between elasticity and strength (G′=stress/strain) and, as such, provides a quantitative measurement of a composition's hardness or softness. Although depending on the speed at which the force is applied, a stiffer composition will have a higher elastic modulus and it will take a greater force to deform the material a given distance, such as, e.g., an injection.

Viscous modulus is also known as the loss modulus because it describes the energy that is lost as viscous dissipation. Tan δ is the ratio of the viscous modulus and the elastic modulus, tan δ=G″/G′. Falcone, supra, 2009. For tan δ values disclosed in the present specification, a tan δ is obtained from the dynamic modulus at a frequency of 0.628 rad/s. A lower tan δ corresponds to a stiffer, harder, or more elastic composition.

Thus, in an embodiment, a hydrogel composition comprising crosslinked glycosaminoglycan polymers as disclosed herein exhibits a complex modulus. In aspects of this embodiment, a hydrogel composition exhibits a complex modulus of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, or about 800 Pa. In other aspects of this embodiment, a hydrogel composition exhibits a complex modulus of, e.g., at most 25 Pa, at most 50 Pa, at most 75 Pa, at most 100 Pa, at most 125 Pa, at most 150 Pa, at most 175 Pa, at most 200 Pa, at most 250 Pa, at most 300 Pa, at most 350 Pa, at most 400 Pa, at most 450 Pa, at most 500 Pa, at most 550 Pa, at most 600 Pa, at most 650 Pa, at most 700 Pa, at most 750 Pa, or at most 800 Pa. In yet other aspects of this embodiment, a hydrogel composition exhibits a complex modulus of, e.g., about 25 Pa to about 150 Pa, about 25 Pa to about 300 Pa, about 25 Pa to about 500 Pa, about 25 Pa to about 800 Pa, about 125 Pa to about 300 Pa, about 125 Pa to about 500 Pa, or about 125 Pa to about 800 Pa.

In another embodiment, a hydrogel composition comprising crosslinked glycosaminoglycan polymers as disclosed herein exhibits an elastic modulus. In aspects of this embodiment, a hydrogel composition exhibits an elastic modulus of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, about 800 Pa, about 850 Pa, about 900 Pa, about 950 Pa, about 1,000 Pa, about 1,200 Pa, about 1,300 Pa, about 1,400 Pa, about 1,500 Pa, about 1,600 Pa, about 1700 Pa, about 1800 Pa, about 1900 Pa, about 2,000 Pa, about 2,100 Pa, about 2,200 Pa, about 2,300 Pa, about 2,400 Pa, or about 2,500 Pa.

In another embodiment, a hydrogel composition comprising crosslinked glycosaminoglycan polymers as disclosed herein exhibits a viscous modulus. In aspects of this embodiment, a hydrogel composition exhibits a viscous modulus of, e.g., about 10 Pa to about 700 Pa, for example, about 20 Pa, about 30 Pa, about 40 Pa, about 50 Pa, about 60 Pa, about 70 Pa, about 80 Pa, about 90 Pa, about 100 Pa, about 150 Pa, about 200 Pa, about 250 Pa, about 300 Pa, about 350 Pa, about 400 Pa, about 450 Pa, about 500 Pa, about 550 Pa, about 600 Pa, about 650 Pa, or about 700 Pa. In another embodiment, a hydrogel composition comprising crosslinked glycosaminoglycan polymers as disclosed herein exhibits a hardness. In aspects of this embodiment, a hydrogel composition exhibits a hardness of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, or about 800 Pa. In other aspects of this embodiment, a hydrogel composition exhibits a hardness of, e.g., at least 25 Pa, at least 50 Pa, at least 75 Pa, at least 100 Pa, at least 125 Pa, at least 150 Pa, at least 175 Pa, at least 200 Pa, at least 250 Pa, at least 300 Pa, at least 350 Pa, at least 400 Pa, at least 450 Pa, at least 500 Pa, at least 550 Pa, at least 600 Pa, at least 650 Pa, at least 700 Pa, at least 750 Pa, or at least 800 Pa. In yet other aspects of this embodiment, a hydrogel composition exhibits a hardness of, e.g., about 100 Pa to about 150 Pa, about 100 Pa to about 300 Pa, about 100 Pa to about 500 Pa, about 100 Pa to about 800 Pa, about 125 Pa to about 300 Pa, about 125 Pa to about 500 Pa, or about 125 Pa to about 800 Pa.

In another embodiment, a hydrogel composition comprising crosslinked glycosaminoglycan polymers as disclosed herein exhibits a tan δ. In aspects of this embodiment, a hydrogel composition exhibits a tan δ of, e.g., about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5. In other aspects of this embodiment, a hydrogel composition exhibits a tan δ of, e.g., at most 0.1, at most 0.2, at most 0.3, at most 0.4, at most 0.5, at most 0.6, at most 0.7, at most 0.8, at most 0.9, at most 1.0, at most 1.1, at most 1.2, at most 1.3, at most 1.4, at most 1.5, at most 1.6, at most 1.7, at most 1.8, at most 1.9, at most 2.0, at most 2.1, at most 2.2, at most 2.3, at most 2.4, or at most 2.5. In yet other aspects of this embodiment, a hydrogel composition exhibits a tan δ of, e.g., about 0.1 to about 0.3, about 0.3 to about 0.5, about 0.5 to about 0.8, about 1.1 to about 1.4, about 1.4 to about 1.7, about 0.3 to about 0.6, about 0.1 to about 0.5, about 0.5 to about 0.9, about 0.1 to about 0.6, about 0.1 to about 1.0, about 0.5 to about 1.5, about 1.0 to about 2.0, or about 1.5 to about 2.5.

Aspects of the present specification provide, in part, a hydrogel composition comprising crosslinked glycosaminoglycan polymers as disclosed herein that exhibits a dynamic viscosity. Viscosity is resistance of a fluid to shear or flow caused by either shear stress or tensile stress. Viscosity describes a fluid's internal resistance to flow caused by intermolecular friction exerted when layers of fluids attempt to slide by one another and may be thought of as a measure of fluid friction. The less viscous the fluid, the greater its ease of movement (fluidity).

Viscosity can be defined in two ways; dynamic viscosity (μ, although η is sometimes used) or kinematic viscosity (ν). Dynamic viscosity, also known as absolute or complex viscosity, is the tangential force per unit area required to move one horizontal plane with respect to the other at unit velocity when maintained a unit distance apart by the fluid. The SI physical unit of dynamic viscosity is the Pascal-second (Pa·s), which is identical to N·m−2·s. Dynamic viscosity can be expressed as τ=μdvx/dz, where τ=shearing stress, μz=dynamic viscosity, and dvx/dz is the velocity gradient over time. For example, if a fluid with a viscosity of one Pa·s is placed between two plates, and one plate is pushed sideways with a shear stress of one Pascal, it moves a distance equal to the thickness of the layer between the plates in one second. Dynamic viscosity symbolize by is also used, is measured with various types of rheometers, devices used to measure the way in which a liquid, suspension or slurry flows in response to applied forces.

Kinematic viscosity (ν) is the ratio of dynamic viscosity to density, a quantity in which no force is involved and is defined as follows: ν=μ/ρ, where μ is the dynamic viscosity ρ is density with the SI unit of kg/m³. Kinematic viscosity is usually measured by a glass capillary viscometer as has an SI unit of m²/s.

The viscosity of a material is highly temperature dependent and for either dynamic or kinematic viscosity to be meaningful, the reference temperature must be quoted. For the viscosity values disclosed herein, a dynamic viscosity is measured at 1 Pa with a cone/plane geometry 2°/40 cm and a temperature of 20° C. Examples of the dynamic viscosity of various fluids at 20° C. is as follows: water is about 1.0×10⁻³ Pa·s, blood is about 3-4×10⁻³ Pa·s, vegetable oil is about 60-85×10⁻³ Pa·s, motor oil SE 30 is about 0.2 Pa·s, glycerin is about 1.4 Pa·s, maple syrup is about 2-3 Pa·s, honey is about 10 Pa·s, chocolate syrup is about 10-25 Pa·s, peanut butter is about 150-250 Pa·s, lard is about 1,000 Pa·s, vegetable shortening is about 1,200 Pa·s, and tar is about 30,000 Pa·s.

In aspects of this embodiment, a hydrogel composition comprising crosslinked glycosaminoglycan polymers as disclosed herein exhibits a dynamic viscosity of, e.g., about 10 Pa·s, about 20 Pa·s, about 30 Pa·s, about 40 Pa·s, about 50 Pa·s, about 60 Pa·s, about 70 Pa·s, about 80 Pa·s, about 90 Pa·s, about 100 Pa·s, about 125 Pa·s, about 150 Pa·s, about 175 Pa·s, about 200 Pa·s, about 225 Pa·s, about 250 Pa·s, about 275 Pa·s, about 300 Pa·s, about 400 Pa·s, about 500 Pa·s, about 600 Pa·s, about 700 Pa·s, about 750 Pa·s, about 800 Pa·s, about 900 Pa·s, about 1,000 Pa·s, about 1,100 Pa·s, or about 1,200 Pa·s. In other aspects of this embodiment, a hydrogel composition comprising crosslinked glycosaminoglycan polymers as disclosed herein exhibits a dynamic viscosity of, e.g., at most 10 Pa·s, at most 20 Pa·s, at most 30 Pa·s, at most 40 Pa·s, at most 50 Pa·s, at most 60 Pa·s, at most 70 Pa·s, at most 80 Pa·s, at most 90 Pa·s, at most 100 Pa·s, at most 125 Pa·s, at most 150 Pa·s, at most 175 Pa·s, at most 200 Pa·s, at most 225 Pa·s, at most 250 Pa·s, at most 275 Pa·s, at most 300 Pa·s, at most 400 Pa·s, at most 500 Pa·s, at most 600 Pa·s, at most 700 Pa·s, at most 750 Pa·s, at most 800 Pa·s, at most 900 Pa·s, or at most 1000 Pa·s. In yet other aspects of this embodiment, a hydrogel composition comprising crosslinked glycosaminoglycan polymers as disclosed herein exhibits a dynamic viscosity of, e.g., about 10 Pa·s to about 100 Pa·s, about 10 Pa·s to about 150 Pa·s, about 10 Pa·s to about 250 Pa·s, about 50 Pa·s to about 100 Pa·s, about 50 Pa·s to about 150 Pa·s, about 50 Pa·s to about 250 Pa·s, about 100 Pa·s to about 500 Pa·s, about 100 Pa·s to about 750 Pa·s, about 100 Pa·s to about 1,000 Pa·s, about 100 Pa·s to about 1,200 Pa·s, about 300 Pa·s to about 500 Pa·s, about 300 Pa·s to about 750 Pa·s, about 300 Pa·s to about 1,000 Pa·s, or about 300 Pa·s to about 1,200 Pa·s.

Aspects of the present specification provide, in part, a hydrogel composition comprising crosslinked glycosaminoglycan polymers as disclosed herein that is injectable. As used herein, the term “injectable” refers to a material having the properties necessary to administer the composition into a soft tissue part, area and/or region of an individual using an injection device with a fine needle. As used herein, the term “fine needle” refers to a needle that is 22 gauge or smaller.

In aspect of this embodiment, a hydrogel composition comprising crosslinked glycosaminoglycan polymers as disclosed herein is injectable through a fine needle. In other aspects of this embodiment, a hydrogel composition comprising crosslinked glycosaminoglycan polymers as disclosed herein is injectable through a needle of, e.g., about 22 gauge, about 27 gauge, about 30 gauge, or about 32 gauge. In yet other aspects of this embodiment, a hydrogel composition comprising crosslinked glycosaminoglycan polymers as disclosed herein is injectable through a needle of, e.g., 22 gauge or smaller, 27 gauge or smaller, 30 gauge or smaller, or 32 gauge or smaller. In still other aspects of this embodiment, a hydrogel composition comprising crosslinked glycosaminoglycan polymers as disclosed herein is injectable through a needle of, e.g., about 22 gauge to about 32 gauge, about 22 gauge to about 27 gauge, or about 27 gauge to about 32 gauge.

In aspects of this embodiment, a hydrogel composition comprising crosslinked glycosaminoglycan polymers as disclosed herein can be injected through a 27 gauge needle with an extrusion force of about 60 N, about 55 N, about 50 N, about 45 N, about 40 N, about 35 N, about 30 N, about 25 N, about 20 N, about 15 N, or about 10 N. In other aspects of this embodiment, a hydrogel composition comprising crosslinked glycosaminoglycan polymers as disclosed herein can be injected through a 27 gauge needle with an extrusion force of about 60 N or less, about 55 N or less, about 50 N or less, about 45 N or less, about 40 N or less, about 35 N or less, about 30 N or less, about 25 N or less, about 20 N or less, about 15 N or less, about 10 N or less, or about 5 N or less.

In aspects of this embodiment, a hydrogel composition comprising crosslinked glycosaminoglycan polymers as disclosed herein can be injected through a 32 gauge needle with an extrusion force of about 60 N, about 55 N, about 50 N, about 45 N, about 40 N, about 35 N, about 30 N, about 25 N, about 20 N, about 15 N, or about 10 N. In other aspects of this embodiment, a hydrogel composition comprising crosslinked glycosaminoglycan polymers as disclosed herein can be injected through a 32 gauge needle with an extrusion force of about 60 N or less, about 55 N or less, about 50 N or less, about 45 N or less, about 40 N or less, about 35 N or less, about 30 N or less, about 25 N or less, about 20 N or less, about 15 N or less, about 10 N or less, or about 5 N or less.

Aspects of the present specification provide, in part, a hydrogel composition comprising crosslinked glycosaminoglycan polymers as disclosed herein that exhibits cohesivity. Cohesivity, also referred to as cohesion cohesive attraction, cohesive force, or compression force is a physical property of a material, caused by the intermolecular attraction between like-molecules within the material that acts to unite the molecules. Cohesivity is expressed in terms of grams-force (gmf). A composition should be sufficiently cohesive as to remain localized to a site of administration. Additionally, in certain applications, a sufficient cohesiveness is important for a composition to retain its shape, and thus functionality, in the event of mechanical load cycling. As such, in one embodiment, a hydrogel composition comprising crosslinked glycosaminoglycan polymers as disclosed herein exhibits cohesivity, on par with water. In yet another embodiment, a hydrogel composition comprising crosslinked glycosaminoglycan polymers as disclosed herein exhibits sufficient cohesivity to remain localized to a site of administration. In still another embodiment, a hydrogel composition comprising crosslinked glycosaminoglycan polymers as disclosed herein exhibits sufficient cohesivity to retain its shape. In a further embodiment, a hydrogel composition comprising crosslinked glycosaminoglycan polymers as disclosed herein exhibits sufficient cohesivity to retain its shape and functionality.

In aspects of this embodiment, a hydrogel composition comprising crosslinked glycosaminoglycan polymers as disclosed herein has a cohesivity of, e.g., about 10 gmf, about 20 gmf, about 30 gmf, about 40 gmf, about 50 gmf, about 60 gmf, about 70 gmf, about 80 gmf, about 90 gmf, about 100 gmf, about 150 gmf, or about 200 gmf. In other aspects of this embodiment, a hydrogel composition comprising crosslinked glycosaminoglycan polymers as disclosed herein has a cohesivity of, e.g., at least 10 gmf, at least 20 gmf, at least 30 gmf, at least 40 gmf, at least 50 gmf, at least 60 gmf, at least 70 gmf, at least 80 gmf, at least 90 gmf, at least 100 gmf, at least 150 gmf, or at least 200 gmf. In yet other aspects of this embodiment, a hydrogel composition comprising crosslinked glycosaminoglycan polymers as disclosed herein has a cohesivity of, e.g., at most 10 gmf, at most 20 gmf, at most 30 gmf, at most 40 gmf, at most 50 gmf, at most 60 gmf, at most 70 gmf, at most 80 gmf, at most 90 gmf, at most 100 gmf, at most 150 gmf, or at most 200 gmf. In yet other aspects of this embodiment, a hydrogel composition comprising crosslinked glycosaminoglycan polymers as disclosed herein has a cohesivity of, e.g., about 50 gmf to about 150 gmf, about 60 gmf to about 140 gmf, about 70 gmf to about 130 gmf, about 80 gmf to about 120 gmf, or about 90 gmf to about 110 gmf.

In yet other aspects of this embodiment, a hydrogel composition comprising crosslinked glycosaminoglycan polymers as disclosed herein has a cohesivity of, e.g., about 10 gmf to about 50 gmf, about 25 gmf to about 75 gmf, about 50 gmf to about 150 gmf, about 100 gmf to about 200 gmf, about 100 gmf to about 300 gmf, about 100 gmf to about 400 gmf, about 100 gmf to about 500 gmf, about 200 gmf to about 300 gmf, about 200 gmf to about 400 gmf, about 200 gmf to about 500 gmf, about 200 gmf to about 600 gmf, about 200 gmf to about 700 gmf, about 300 gmf to about 400 gmf, about 300 gmf to about 500 gmf, about 300 gmf to about 600 gmf, about 300 gmf to about 700 gmf, about 300 gmf to about 800 gmf, about 400 gmf to about 500, about 400 gmf to about 600, about 400 gmf to about 700, about 400 gmf to about 800, about 500 gmf to about 600 gmf, about 500 gmf to about 700 gmf, about 500 gmf to about 800 gmf, about 600 gmf to about 700 gmf, about 600 gmf to about 800 gmf, about 700 gmf to about 800 gmf, about 1000 gmf to about 2000 gmf, about 1000 gmf to about 3000 gmf, or about 2000 gmf to about 3000 gmf.

Aspects of the present specification provide, in part, a hydrogel composition comprising crosslinked glycosaminoglycan polymers as disclosed herein that exhibits a physiologically-acceptable osmolarity. As used herein, the term “osmolarity” refers to the concentration of osmotically active solutes in solution. As used herein, the term “a physiologically-acceptable osmolarity” refers to an osmolarity in accord with, or characteristic of, the normal functioning of a living organism. As such, administration of a hydrogel composition as disclosed herein exhibits an osmolarity that has substantially no long term or permanent detrimental effect when administered to a mammal. Osmolarity is expressed in terms of osmoles of osmotically active solute per liter of solvent (Osmol/L or Osm/L). Osmolarity is distinct from molarity because it measures moles of osmotically active solute particles rather than moles of solute. The distinction arises because some compounds can dissociate in solution, whereas others cannot. The osmolarity of a solution can be calculated from the following expression: Osmol/L=Σφᵢ ηᵢ Cᵢ, where φ is the osmotic coefficient, which accounts for the degree of non-ideality of the solution; η is the number of particles (e.g. ions) into which a molecule dissociates; and C is the molar concentration of the solute; and i is the index representing the identity of a particular solute. The osmolarity of a hydrogel composition disclosed herein can be measured using a conventional method that measures solutions.

In an embodiment, a hydrogel composition comprising crosslinked glycosaminoglycan polymers as disclosed herein exhibits a physiologically-acceptable osmolarity. In aspects of this embodiment, a hydrogel composition exhibits an osmolarity of, e.g., about 100 mOsm/L, about 150 mOsm/L, about 200 mOsm/L, about 250 mOsm/L, about 300 mOsm/L, about 350 mOsm/L, about 400 mOsm/L, about 450 mOsm/L, or about 500 mOsm/L. In other aspects of this embodiment, a hydrogel composition exhibits an osmolarity of, e.g., at least 100 mOsm/L, at least 150 mOsm/L, at least 200 mOsm/L, at least 250 mOsm/L, at least 300 mOsm/L, at least 350 mOsm/L, at least 400 mOsm/L, at least 450 mOsm/L, or at least 500 mOsm/L. In yet other aspects of this embodiment, a hydrogel composition exhibits an osmolarity of, e.g., at most 100 mOsm/L, at most 150 mOsm/L, at most 200 mOsm/L, at most 250 mOsm/L, at most 300 mOsm/L, at most 350 mOsm/L, at most 400 mOsm/L, at most 450 mOsm/L, or at most 500 mOsm/L. In still other aspects of this embodiment, a hydrogel composition exhibits an osmolarity of, e.g., about 100 mOsm/L to about 500 mOsm/L, about 200 mOsm/L to about 500 mOsm/L, about 200 mOsm/L to about 400 mOsm/L, about 300 mOsm/L to about 400 mOsm/L, about 270 mOsm/L to about 390 mOsm/L, about 225 mOsm/L to about 350 mOsm/L, about 250 mOsm/L to about 325 mOsm/L, about 275 mOsm/L to about 300 mOsm/L, or about 285 mOsm/L to about 290 mOsm/L. In a specific embodiment, the compositions exhibit an osmolarity of between about 270 mOsm/L and about 390 mOsm/L. In one embodiment the compositions have an osmolarity of about 300 mOsm/L, more specifically, 308 mOsm/L.

Aspects of the present specification provide, in part, a hydrogel composition comprising crosslinked glycosaminoglycan polymers as disclosed herein that exhibits a physiologically-acceptable osmolality. As used herein, the term “osmolality” refers to the concentration of osmotically active solutes per kilo of solvent in the body. As used herein, the term “a physiologically-acceptable osmolality” refers to an osmolality in accord with, or characteristic of, the normal functioning of a living organism. As such, administration of a hydrogel composition disclosed herein exhibits an osmolality that has substantially no long term or permanent detrimental effect when administered to a mammal. Osmolality is expressed in terms of osmoles of osmotically active solute per kilogram of solvent (osmol/kg or Osm/kg) and is equal to the sum of the molalities of all the solutes present in that solution. The osmolality of a solution can be measured using an osmometer. The most commonly used instrument in modern laboratories is a freezing point depression osmometer. This instruments measure the change in freezing point that occurs in a solution with increasing osmolality (freezing point depression osmometer) or the change in vapor pressure that occurs in a solution with increasing osmolality (vapor pressure depression osmometer).

In an embodiment, a hydrogel composition comprising crosslinked glycosaminoglycan polymers as disclosed herein exhibits a physiologically-acceptable osmolality. In aspects of this embodiment, a hydrogel composition exhibits an osmolality of, e.g., about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, or about 500 mOsm/kg. In other aspects of this embodiment, a hydrogel composition exhibits an osmolality of, e.g., at least 100 mOsm/kg, at least 150 mOsm/kg, at least 200 mOsm/kg, at least 250 mOsm/kg, at least 300 mOsm/kg, at least 350 mOsm/kg, at least 400 mOsm/kg, at least 450 mOsm/kg, or at least 500 mOsm/kg. In yet other aspects of this embodiment, a hydrogel composition exhibits an osmolality of, e.g., at most 100 mOsm/kg, at most 150 mOsm/kg, at most 200 mOsm/kg, at most 250 mOsm/kg, at most 300 mOsm/kg, at most 350 mOsm/kg, at most 400 mOsm/kg, at most 450 mOsm/kg, or at most 500 mOsm/kg. In still other aspects of this embodiment, a hydrogel composition exhibits an osmolality of, e.g., about 100 mOsm/kg to about 500 mOsm/kg, about 200 mOsm/kg to about 500 mOsm/kg, about 200 mOsm/kg to about 400 mOsm/kg, about 300 mOsm/kg to about 400 mOsm/kg, about 270 mOsm/kg to about 390 mOsm/kg, about 225 mOsm/kg to about 350 mOsm/kg, about 250 mOsm/kg to about 325 mOsm/kg, about 275 mOsm/kg to about 300 mOsm/kg, or about 285 mOsm/kg to about 290 mOsm/kg.

### Example 1

### Synthesis of a Multifunctional PEG-Based Crosslinking Agent

This example illustrates how to make a multifunctional PEG-based crosslinking agent useful in the present injectable devices, from a base polyalcohol.

A multifunctional PEG-based crosslinking agent such as disclosed elsewhere herein can be synthesized using a general scheme below. A base polyalcohol of about 200 Da to about 10,000 Da, and having the desired length and branching, is initially reacted with sodium hydride or any other reagent that can deprotonate the hydroxyl groups and then with epichlorohydrin or any other appropriate epoxide group(s). In the schematic below, a 4-arm base alcohol is shown; where n may be an integer of 0 to 60. In addition, although the general chemical schematic is illustrated with a 4-arm base polyalchohol, a similar synthesis scheme is employed to produce other multifunctional PEG-based crosslinking agents by simply using the appropriate base polyalcohol. For example, to synthesize a bifunctional PEG-based crosslinker, a 2-arm base polyalcohol is used; to synthesis a trifunctional PEG-based crosslinker, a 3-arm base polyalcohol is used; to synthesis a pentafunctional PEG-based crosslinker, a 5-arm base polyalcohol is used; to synthesis a hexafunctional PEG-based crosslinker, a 6-arm base polyalcohol is used; to synthesis a heptafunctional PEG-based crosslinker, a 7-arm base polyalcohol is used; to synthesis an octafunctional PEG-based crosslinker, a 8-arm base polyalcohol is used; to synthesis a nonafunctional PEG-based crosslinker, a 9-arm base polyalcohol is used; to synthesis a decafunctional PEG-based crosslinker, a 10-arm base polyalcohol is used; etc.

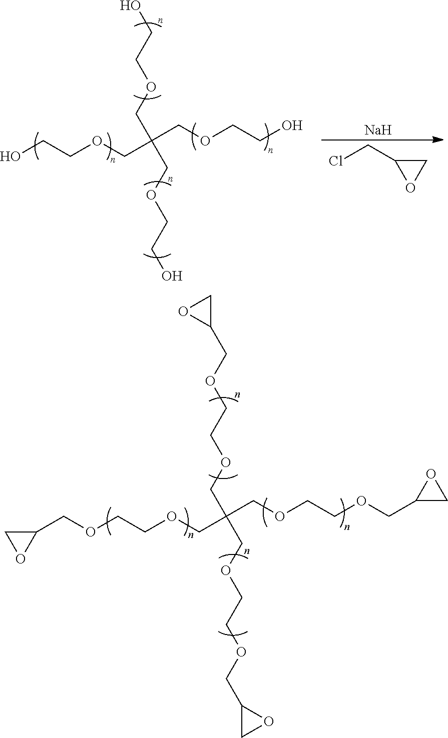

To synthesize pentaerythritol tetraglycidyl ether, 136 mg of pentaerythritol was reacted with 100 mg of sodium hydride and subsequently with 370 mg of epichlorohydrin.

### Example 2

### Crosslinking of Glycosaminoglycan Polymers Using Multifunctional PEG-Based Crosslinker

This example illustrates how to crosslink glycosaminoglycan polymers using a multifunctional PEG-based crosslinking agent as disclosed herein.

To crosslink glycosaminoglycan polymers using a multifunctional PEG-based crosslinker, 400 mg of low molecular weight sodium hyaluronate, such as, e.g., about 400,000 Da, was mixed with 2.3 grams of 1% sodium hydroxide solution and hydrated by incubating at ambient temperature for about 30 minutes. Alternatively, a high molecular weight sodium hyaluronate, such as, e.g., about 2,000,000 Da can be used. After hydration, about 80 mg (20% w/w) of a tetrafunctional PEG-based crosslinking agent of Example 1 (about 360 Da) was added to the hydrated sodium hyaluronate. The mixture was then mechanically homogenized, and then placed in an about 50° C. oven for about 90 minutes. The resulting crosslinked hydrogel is neutralized with an equimolar amount of hydrochloric acid and swelled in a phosphate buffer (pH 7.4). The resulting hydrogel comprising crosslinked hyaluronan polymers was processed once through a 60 μm mesh and dialyzed for one week using a 20 kDa MWCO bag. The dialyzed hydrogel was then transferred to 0.8 mL syringe and flash sterilized at 128° C.

A hydrogel composition as disclosed herein was alternatively produced as described above, except that after hydration, about 56 mg (14% w/w) of a tetrafunctional PEG-based crosslinking agent of Example 1 (about 360 Da) was added to the hydrated sodium hyaluronate.

To crosslink glycosaminoglycan polymers using a multifunctional PEG-based crosslinker and another non-PEG-based crosslinker, 0.4 g of sodium hyaluronate (about 400,000 Da) was mixed with 2.3 grams of 1% sodium hydroxide solution and hydrated by incubating at ambient temperature for about 30 minutes. After hydration, about 20 mg of 1,4-butanediol diglycidyl ether (BDDE) and about 80 mg of a tetrafunctional PEG-based crosslinking agent of Example 1 (about 360 Da) were added to the hydrated sodium hyaluronate. The mixture was then mechanically homogenized, and then placed in a 50° C. oven for about 90 minutes. The resulting crosslinked hydrogel is neutralized with an equimolar amount of hydrochloric acid and swelled in a phosphate buffer (pH7.4).

### Example 2A

### Crosslinking of Glycosaminoglycan Polymers Using Multifunctional PEG-Based Crosslinker

This is an example of how to make a glycosaminoglycan polymer hydrogel using a multifunctional PEG-based crosslinking agent as disclosed herein.

About 60 mg of low molecular weight sodium hyaluronate, such as, e.g., about (310,000 Da-840,000 Da), was hydrated in an appropriate amount of NaOH 0.25N for about 1 hour and homogenized by cartridge/cartridge mixing.

After hydration, a sufficient amount of a pentaerythrithol tetra glycidyl ether crosslinking agent (about 13% w/w) was added to the hydrated sodium hyaluronate and the mixture homogenized by cartridge/cartridge mixing and then placed in an about 50° C. oven for about 120 minutes. At this step, hydrogel had a NaHA concentration of about 135 mg/g.

The resulting crosslinked hydrogel was neutralized in a solution made of HCl 1N/Phosphate Buffer and swollen (less than 24H) to reach a NaHA concentration of 30 mg/g.

The resulting hydrogel comprising crosslinked hyaluronan polymers was processed once through a 100 μm mesh filter and dialyzed for about 30-50 hours against a phosphate buffer to reach a concentration of 25 mg/g in NaHA and to remove residual crosslinker. An amount of lidocaine hydrochloride was added to the hydrogel to reach a lidocaine concentration of 0.3% w/w.

The dialyzed hydrogel was then transferred to 0.8 mL COC (cyclo olefin copolymer) syringes and steam sterilized.

### Example 3

### Rheological Behavior of Compositions of the Invention

Rheological behavior of three test batch compositions in accordance with the invention, were evaluated as follows. The compositions are 100% low molecular weight hyaluronic acid (Na-HA) crosslinked with 13% of pentaerythritol tetra glycidyl ether (PEGE)/NaHA (w/w), formulated to a concentration of about 25 mg/g with 0.3% lidocaine hydrochloride (w/w) in a phosphate buffer.

Procedure: Measurements were performed at a constant temperature of 25.0±0.1° C. with a 40 mm cone with an angle of 2°. Oscillatory shear tests were performed over a range of frequencies from 0.05 Hz to 10 Hz, using a constant strain of 8.10⁻³.

Instrument: Rheometer RS600 (G85), RS6000 (G156) and Mars 3 (G163).

Material: 2-3 syringes containing 1 ml of a composition (Test Batch 1, Test Batch 2, and Test Batch 3) in accordance with the invention, left at ambient temperature at least 2 hours before the measurements, were tested. For each frequency, results indicated in Table 1 are the mean value of these 2 measurements.

TABLE 1
Rheological characteristics

| | | | Test | Test | |
|---|---|---|---|---|---|
| | | Test Batch 1* | Batch 2 | Batch 3 | Average |
| 0.01 Hz | G′ (Pa) | 2078 | 1894 | 1850 | 1941 +/− 121 |
| | G″ (Pa) | 311 | 314 | 287 | 304 +/− 15 |
| 1 Hz | G′ (Pa) | 2449 | 2285 | 2190 | 2308 +/− 131 |
| | G″ (Pa) | 384 | 386 | 358 | 376 +/− 16 |
| 5 Hz | G′ (Pa) | 2846 | 2655 | 2510 | 2670 +/− 169 |
| | G″ (Pa) | 325 | 336 | 297 | 319 +/− 10 |
| 10 Hz | G′ (Pa) | 2949 | 2779 | 2630 | 2786 +/− 16 |
| | G″ (Pa) | 297 | 286 | 256 | 280 +/− 21 |

*average of 3 syringes

G′ (elastic modulus) and G″ (viscous modulus) describe, respectively, the stiffness and viscous resistance to deformation of the material and are measured in order to quantify the elastic and viscous nature of the materials. G′ was consistently higher than G″ for STAR-HA, indicating that the elastic properties of the materials, dominate the viscous properties. There was no crossover point seen in the range of frequencies studied. Results, indicated in Table 1, show the dominance of elastic properties with G′>G″ (and consequently tan δ<1) for all the range of frequencies studied. Average values of 2308±131 Pa and 376±16 Pa, for G′ and G″ respectively, were found at 1 Hz for the compositions of the invention.

### Example 4

### Injectable Devices of the Invention Used to Correct a Recessed or Retruded Chin

About 3 ml of an injectable device in accordance with the invention, comprising an HA-based formulation or composition, for facial augmentation and contouring, is administered by injection through a 25 gauge cannula, (other suitable gauges are useful as well, for example, 22-28 gauge cannulas or needles) supraperiostally, into a chin of a 26 year old male patient suffering from a retruded chin. The composition comprises 100% low molecular weight hyaluronic acid (Na-HA) crosslinked with 12% pentaerythritol tetra glycidyl ether (PEGE)/NaHA (w/w), formulated to a concentration of about 27 mg/g with 0.3% lidocaine hydrochloride (w/w) in a phosphate buffer. The physician carefully injects the composition in a manner so as to provide lift and desirable shape to the chin. 12 months after the procedure, the patient is still satisfied with the results and his chin remains “strong” and appears more masculine. 18 months after the procedure, there is no visible reduction of his chin and he remains satisfied with the results.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An injectable device for augmenting the chin in a human being, the device comprising:
a composition comprising a hyaluronic acid crosslinked with a multifunctional polyethylene glycol (PEG)-based crosslinking agent.

2. The device of claim 1 wherein the hyaluronic acid is crosslinked with between about 10% and about 20% pentaerythritol tetra glycidyl ether (PEGE)/NaHA (w/w).

3. The device of claim 1 wherein the composition is crosslinked with about 13% of pentaerythritol tetra glycidyl ether (PEGE)/NaHA (w/w).

4. The device of claim 1 wherein the hyaluronic acid, prior to crosslinking, is substantially entirely a low molecular weight hyaluronic acid.

5. The device of claim 1 wherein the tetrafunctional crosslinker is pentaerythritol tetraglycidyl ester.

6. The device of claim 1 wherein the composition has an HA concentration of between about 20 mg/g and about 30 mg/g.

7. The device of claim 1 wherein the composition has an HA concentration of about 25 mg/g.

8. The device of claim 1 wherein the composition is made by a process comprising crosslinking a low molecular weight hyaluronic acid with a multifunctional polyethylene glycol (PEG)-based crosslinking agent.

9. The device of claim 1 wherein the composition further comprises a lidocaine.

10. The device of claim 1 wherein the composition comprises about 0.3% lidocaine HCL w/w.

11. The device of claim 1 wherein the composition exhibits an osmolarity of between about 270 mOsm/L and about 390 mOsm/L.

12. The device of claim 1 wherein the hyaluronic acid has a mean molecular weight of between about 300,000 Da and about 900,000 Da.

13. The device of claim 1 wherein the hyaluronic acid has a mean molecular weight of about 650,000 Da.

14. A method for correcting chin retrusion in a patient comprising:
supraperiostally administering in the chin of the patient, an effective amount of a composition comprising a low molecular weight hyaluronic acid and a PEG-based, non-BDDE crosslinker.

15. The method of claim 14 wherein the composition is crosslinked with between about 10% and about 20% pentaerythritol tetra glycidyl ether (PEGE)/NaHA (w/w).

16. The method of claim 14 wherein the composition is crosslinked with about 13% of pentaerythritol tetra glycidyl ether (PEGE)/NaHA (w/w).

17. The method of claim 14 wherein the hyaluronic acid, prior to crosslinking, is substantially entirely a low molecular weight hyaluronic acid.

18. The method of claim 14 wherein the tetrafunctional crosslinker is pentaerythritol tetraglycidyl ester.

19. The method of claim 14 wherein the composition has an HA concentration of about 25 mg/g.

20. The method of claim 14 wherein the composition is made by a process comprising crosslinking a low molecular weight hyaluronic acid with a multifunctional polyethylene glycol (PEG)-based crosslinking agent.

21. A method for contouring or correcting a facial feature of a patient, the method comprising:
subdermally administering into the facial feature of the patient, an effective amount of a composition comprising hyaluronic acid crosslinked with pentaerythritol tetraglycidyl ester.

22. The method of claim 21 wherein the facial feature is the chin of the patient.

23. An injectable device for correcting a retruded chin in a human being, the device comprising:
an injectable composition comprising a non-BDDE crosslinked hyaluronic acid comprising about 13% pentaerythritol tetra glycidyl ether (PEGE)/HA (w/w), the hyaluronic acid being substantially entirely a low molecular weight hyaluronic acid having a mean molecular weight of between about 300,000 Da and about 900,000 Da;
the composition having an HA concentration of about 20 mg/g to about 30 mg/g; and
the composition comprising about 0.3% lidocaine w/w; and
wherein the composition exhibits an osmolarity of between about 270 mOsm/L and about 390 mOsm/L.

24. The device of claim 23 wherein said low molecular weight hyaluronic acid has a mean molecular weight of about 650,000 Da.
